(19) United States
(12) Patent Application Publication
(10) Pub. No.: US 2016/0263130 A1
(43) Pub. Date: Sep. 15, 2016

(54) PHARMACEUTICAL COMPOSITION FOR PREVENTING OR TREATING LIVER DISEASES, CONTAINING PLASMALOGEN PRECURSOR, PLASMALOGEN OR PLASMALOGEN ANALOG AS EFFECTIVE COMPONENT

(71) Applicant: UNIVERSITY OF ULSAN FOUNDATION FOR INDUSTRY COOPERATION, Nam-gu Ulsan (KR)

(72) Inventors: Ki-Up LEE, Gyeonggi-do (KR); Eun Hee KOH, Seoul (KR); Jung Eun JANG, Seoul (KR)

(73) Assignee: UNIVERSITY OF ULSAN FOUNDATION FOR INDUSTRY COOPER ATION, Ulsan (KR)

(21) Appl. No.: 15/032,635

(22) PCT Filed: Oct. 23, 2014
(86) PCT No.: PCT/KR2014/010030

§ 371 Date: Apr. 28, 2016

(30) Foreign Application Priority Data
Oct. 29, 2013 (KR) 10-2013-0129213
Oct. 22, 2014 (KR) 10-2014-0143563

(51) Int. Cl. A61K 31/661; A23L 2/52

(52) U.S. Cl. CPC A61K 31/661; A23L 1/3008; A23L 2/52; A23V 2002/00

(57) ABSTRACT

The present invention relates to a pharmaceutical composition for preventing or treating liver diseases, containing a plasmalogen precursor, plasmalogen, or a plasmalogen analog as an effective component. More specifically, a plasmalogen precursor, or plasmalogen or a plasmalogen analog produced by metabolizing the plasmalogen precursor, can prevent or treat liver diseases such as hepatic steatosis, hepatitis, or liver cirrhosis, etc. by decreasing accumulation of neutral fats in the liver, repressing expressions of inflammatory cytokine genes, and increasing fatty acid oxidation capacity of peroxisome.

### TECHNICAL FIELD

The present invention relates to a pharmaceutical composition and a health food composition that are each for preventing or treating liver diseases such as hepatic steatosis, hepatitis, or liver cirrhosis by including a plasmalogen precursor, plasmalogen, or an analog thereof as an effective component.

### BACKGROUND ART

Liver is the organ that plays a key role in nutrient metabolism, weighing about 1,500 g in human subjects. Abnormal liver function may cause problems in the nutrient metabolism, including defects in glycogen synthesis from glucose, converting protein into albumin, or decomposing and delivering unnecessary materials to be excreted through bile.

The most common cause of liver disease is hepatitis, and this can be classified into acute and chronic, and also into viral hepatitis, alcoholic hepatitis, or drug-induced hepatitis depending on the causes. A spectrum of liver disease also includes hepatic steatosis, hepatitis (steatohepatitis), liver cirrhosis, and liver cancer. Mechanisms of progression of the liver diseases have not been completely established, but hepatic steatosis is one of the primary lesions that lead to secondary hepatocyte damage. Thus effective control of hepatic steatosis may prevent occurrence of severe liver diseases. Currently, the liver diseases have been treated by exercise, abstention from alcohol, or dietary therapy, accompanied by drug treatment. However, there has been no treatment method that fundamentally or completely treats liver diseases.

Liver pills prescribed on the market have prevention and treatment effects with respect to acute liver damages in animals or clinical trials in many cases, and the pills include silymarin (e.g., Legalon®), UDCA (e.g., Urusa®), or PMC (Nissel™). However, the pills or any other agents have not yet exhibited significant treatment effects.

Recently, peroxisome, an intracellular organelle, as well as mitochondria have been shown to play an important role in fatty acid oxidation. Abnormal peroxisome function has been reported to cause hepatic steatosis. Also, peroxisome decomposes reactive oxygen species and is essential for synthesis of a material known as plasmalogen.

Plasmalogen belongs to phospholipids that has a vinyl ether bond, and is a main component of a cell layer. Plasmalogen is also known to play an important role in maintaining cell function by acting as a signaling molecule or producing ant-oxidation effects.

Korean Patent No. 10-2011-7015617 discloses that a pharmaceutical composition including a plasmalogen compound is used to treat or prevent membrane cholesterol-related aging diseases such as neurodegeneration, cognitive impairment, osteoporosis, bipolar disorder, and vascular diseases. However, treatment or prevention of liver diseases by using plasmalogen as disclosed in the present invention have not been described.

In this regard, the present inventors have discovered that a plasmalogen precursor, plasmalogen, or a plasmalogen analog suppresses accumulation of fats in the liver and expression of inflammatory genes. We thus completed the present invention to provide a pharmaceutical composition and a health food composition for preventing or treating liver diseases, wherein each of the compositions includes a plasmalogen precursor, plasmalogen, or a plasmalogen analog.

### DETAILED DESCRIPTION OF THE INVENTIVE CONCEPT

### Technical Problem

The present invention provides a pharmaceutical composition and a health food composition, which prevent or treat liver diseases by decreasing fat accumulation in the liver and suppressing expression of genes that causes inflammation in liver tissues, and each of the compositions includes a plasmalogen precursor, plasmalogen, and a plasmalogen analog.

### Technical Solution

The present invention is a pharmaceutical composition and a health food composition, which prevent or treat liver diseases by decreasing accumulation of fats in liver and suppressing expression of genes that causes inflammation in liver tissues. This includes a plasmalogen precursor, plasmalogen, and a plasmalogen analog.

The plasmalogen precursor may be an alkoxy glycerol having a C16-C18 fatty alcohol linked to a glycerol backbone via an ether bond. In particular, the alkoxy glycerol may be selected from the group consisting of a batyl alcohol, a chimyl alcohol, a selachyl alcohol, and a mixture thereof, but is not limited thereto.

More preferably, the alkoxy glycerol may be shark liver oil, and the shark liver oil may include squalene in addition to alkoxy glycerol including batyl alcohol, chimyl alcohol, and selachyl alcohol.

The plasmalogen precursor, plasmalogen, or plasmalogen analog decreases accumulation of neutral fats, and suppresses expression of inflammatory cytokine genes, and increases fatty acids oxidation of peroxisome.

The liver diseases may be selected from the group consisting of hepatic steatosis, hepatitis, and liver cirrhosis.

The invention may contain plasmalogen precursor, plasmalogen, or plasmalogen analog at an amount of 0.01 part to about 90 parts (by weight) of the 100 parts (by weight) of the pharmaceutical agent.

The invention can be provided as a health food composition that includes one or more than two selected forms of a plasmalogen precursor, plasmalogen, and a plasmalogen analog as an effective component.

### Advantageous Effects

According to the present invention, a plasmalogen precursor, or plasmalogen or a plasmalogen analog produced by metabolizing the plasmalogen precursor, can prevent or treat liver diseases such as hepatic steatosis, hepatitis, or liver cirrhosis, etc. by decreasing accumulation of neutral fats in the liver, suppressing expressions of inflammatory cytokine genes, and increasing fatty acid oxidation capacity in peroxisome.

### DESCRIPTION OF THE DRAWINGS

FIG. 1 is a graph showing changes in plasma alanine aminotransferase (ALT) level in a group of experimental mice treated with methionine-choline deficient diet (MCDD), which is an animal model of human nonalcoholic steatohepatitis. A group of experimental mice was treated with MCDD and 100 mg/kg/day of plasmalogen precursor alkoxy glycerol. P***<0.001 vs a control group (CON); P#<0.05 vs MCDD;

FIG. 2 is a graph showing changes in neutral fat amounts in livers of the group of experimental mice treated with MCDD and the group of experimental mice treated with MCDD and 100 mg/kg/day of plasmalogen precursor alkoxy glycerol. P*<0.05 vs CON; P#<0.05 vs MCDD;

FIG. 3 is a graph showing changes in the expression of inflammatory cytokine genes of the group of experimental mice treated with MCDD and the group of experimental mice treated with MCDD and 100 mg/kg/day of plasmalogen precursor alkoxy glycerol. P*<0.05 vs CON; P#<0.05 vs MCDD;

FIG. 4 shows the results of histological change in livers of the group of experimental mice treated with MCDD and the group of experimental mice treated with MCDD and 100 mg/kg/day of plasmalogen precursor alkoxy glycerol;

FIG. 5 is a graph showing mitochondrial fatty acid oxidation (left) and peroxisome fatty acid oxidation (right) as the results of changes in liver tissue fatty acid oxidation capacity of the group of experimental mice treated with MCDD and the group of experimental mice treated with MCDD and 100 mg/kg/day of plasmalogen precursor alkoxy glycerol. P*<0.05 vs CON; P#<0.05 vs MCDD; and

FIG. 6 is a graph that shows the results of live histological change and confirms levels of liver neutral fats and plasma ALT of the group of experimental mice treated with MCDD and a group (AG-200) of experimental mice treated with MCDD and 200 mg/kg/day of plasmalogen precursor alkoxy glycerol.

### BEST MODE

The present invention may provide a pharmaceutical composition for preventing or treating liver diseases, and the composition includes one or more than two selected from the group consisting of a plasmalogen precursor, plasmalogen, and a plasmalogen analog as an effective component.

The plasmalogen precursor may be alkoxy glycerol that has C16-C18 fatty alcohol linked to a glycerol backbone via an ether bond. In particular, the alkoxy glycerol may be selected from the group consisting of batyl alcohol, chimyl alcohol, selachyl alcohol, and a mixture thereof, but is not limited thereto.

More preferably, the alkoxy glycerol may be shark liver oil, and the shark liver oil includes shark liver oil in addition to alkoxy glycerol including batyl alcohol, chimyl alcohol, and selachyl alcohol.

In one embodiment, the plasmalogen or the plasmalogen analog may be orally administered in the form of alkyl glycerol and then converted into the form of plasmalogen or a plasmalogen analog.

The plasmalogen precursor, the plasmalogen, or the plasmalogen analog may decrease accumulation of neutral fats, suppress expression of inflammatory cytokine genes, and increases fatty acid oxidation of peroxisome.

The liver diseases may be selected from the group consisting of hepatic steatosis, hepatitis, and liver cirrhosis.

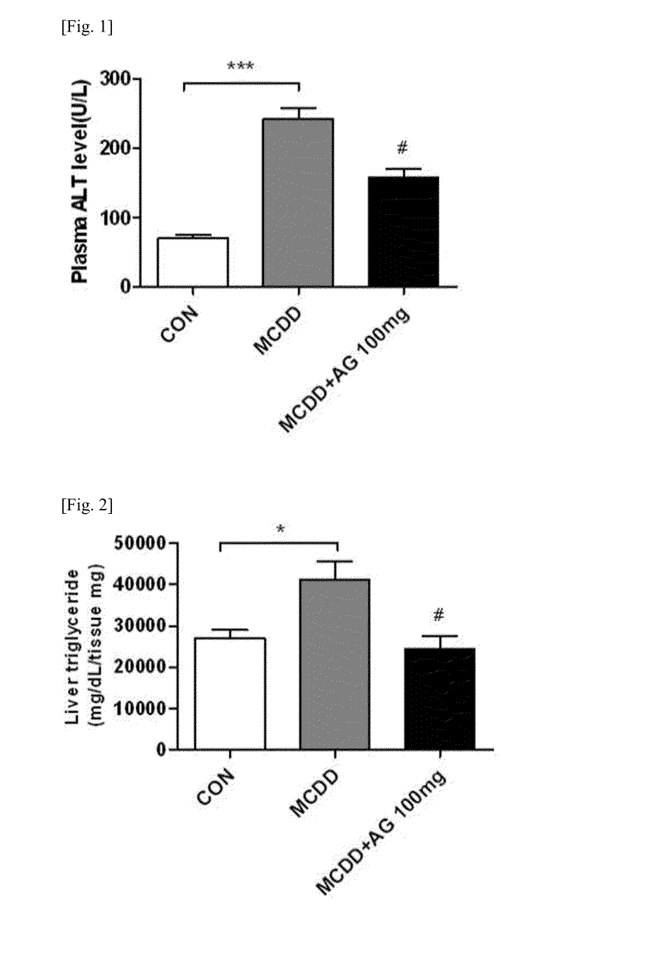

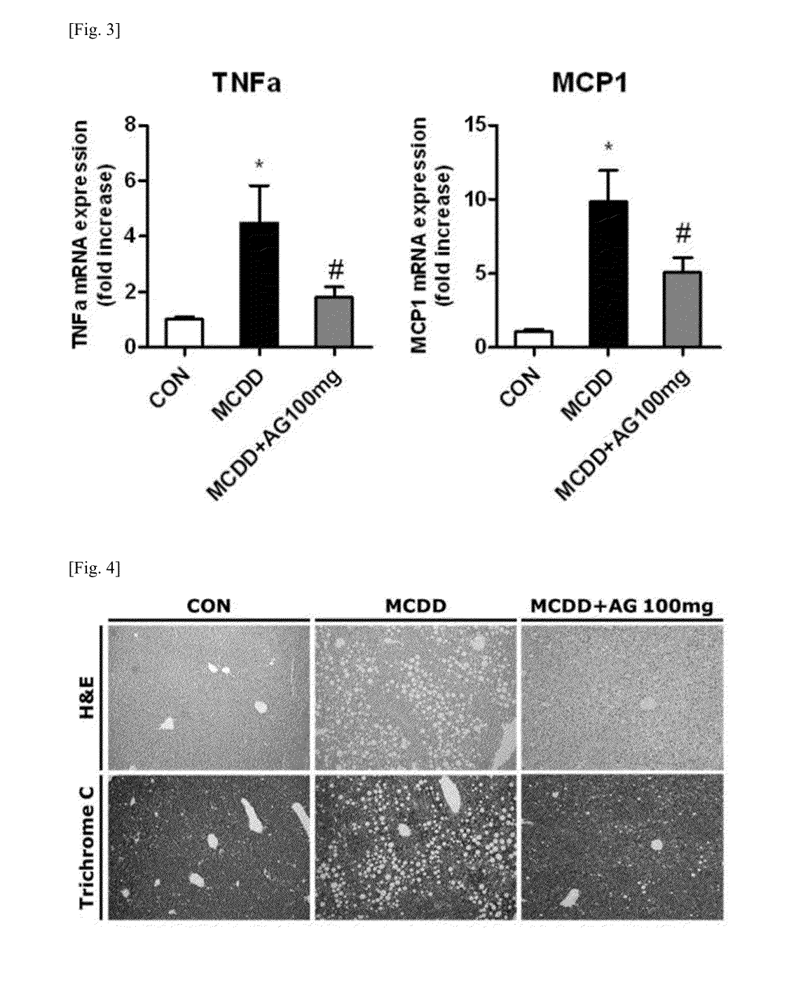

For example, plasma levels of alanine aminotransferase (ALT) were significantly decreased in the animal group given diet that induces hepatic steatohepatitis (i.e., MCDD) and together with alkoxy glycerol, which is a plasmalogen precursor, than the control group given MCDD only (FIG. 1). Real-time gene amplification of the liver tissues, confirmed that expression of inflammatory cytokine genes was suppressed in MCDD+alkoxyl glycerol group as shown in FIG. 3. In this regard, the plasmalogen or an analog thereof produced positive effects in terms of preventing or treating hepatitis and liver cirrhosis.

Also, as a result of confirming neutral fats in the liver by measuring triglyceride, the experimental group administered with the MCDD together with alkoxy glycerol, as a plasmalogen precursor, showed improvement in hepatic steatosis as comparable to the results of a control group administered with normal diet as shown in FIG. 2. In addition, as shown in FIG. 4, the experimental group showed significant improvement in the liver fibrosis. In this regard, it can be concluded that the plasmalogen or the analog thereof produces excellent effects in terms of preventing or treating a hepatic steatosis, stetohepatitis and fibrosis.

The plasmalogen precursor, plasmalogen, or plasmalogen analog may be contained at an amount in a range of about 0.01 part to about 90 parts by weight based on 100 parts by weight of the total weight of the pharmaceutical composition.

In one embodiment of the present invention, a pharmaceutical composition for preventing or treating liver diseases, the composition including the plasmalogen or an analog thereof as an effective component, may be used in any formulation selected from the group consisting of injections, granules, powders, tablets, pills, capsules, suppositories, gels, suspensions, emulsions, drops, or solutions.

In another embodiment of the present invention, a pharmaceutical composition for preventing or treating liver diseases, the composition including the plasmalogen precursor, plasmalogen, or plasmalogen analog as an effective component, may include at least one additive selected from the group consisting of appropriate carriers, excipients, disintegrating agents, sweetening agents, coating agents, swelling agents, glidants, flavors, antioxidants, buffers, bacteriostatic agents, diluents, dispersing agents, surfactants, additives, and lubricants.

In particular, the carriers, excipients, and diluents may be prepared by using lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, or mineral oil. Examples of a solid formulation for oral administration may include tablets, pills, powders, granules, and capsules, and the solid formulation may be prepared by mixing at least one excipient, for example, starch, calcium carbonate, sucrose or lactose, or gelatin to the composition. Also, lubricants such as magnesium stearate or talc, in addition to a simple excipient, may be used. Examples of the solution formulation for oral administration may include suspensions, liquids for internal use, emulsions, or syrups, and various excipients, such as wetting agents, sweetening agents, aromas, and preservatives, may be included in addition to water or liquid paraffin, which is a simple diluent that is commonly used. Examples of a formulation for parenteral administration may include sterilized aqueous solutions, non-aqueous solvents, suspending agents, emulsions, lyophilized preparations, or suppositories. Examples of the non-aqueous solvents or suspending agents may include propylene glycol, polyethylene glycol, vegetable oil such as olive oil, or injectable esters such as ethyloleate. Materials for the suppositories may include witepsol, macrogol, tween 61, cacao paper, laurin paper, glycerogelatin.

According to one embodiment of the present invention, the pharmaceutical composition may be administered to the subject by using a common method via intravenous, intra-arterial, intraperitoneal, intramuscular, intrasternal, percutaneous, endonasal, inhalation, topical, rectal, oral, intraocular, or intradermal routes.

A preferable dose of the plasmalogen precursor, plasmalogen, or plasmalogen analog may differ depending on the condition and weight of the subject, types and degrees of the disease, form of drugs, and routes and periods of the administration, and the dose may be appropriately selected by one of ordinary skill in the art. According to one embodiment of the present invention, the dose may be in a range of, but not limited to, about 0.01 mg/kg to about 200 mg/kg per day, or, for example, about 0.1 mg/kg to about 200 mg/kg per day, or about 0.1 mg/kg to about 100 mg/kg per day. The administration may be performed once a day or several times in a day, but embodiments are not limited thereto.

In the present invention, the ‘subject’ may be a mammal including a human, but is not limited thereto.

According to another embodiment, provided may be a health food composition for improving or preventing liver diseases, and the composition includes one or at least two selected from a plasmalogen precursor, plasmalogen, and a plasmalogen analog as an effective component.

The health food may be provided in the form of powders, granules, tablets, capsules, syrups, or drinks, and the health food may be used together with food other than the plasmalogen precursor, plasmalogen, or plasmalogen analog as an effective component according to the present invention or food additives and may be appropriately used according to a common method. A mixture amount of the effective component may be appropriately determined depending on the purpose of use, for example, for prevention, health, or remedial treatment.

An effective dose of the plasmalogen precursor, plasmalogen, or plasmalogen analog included in the health food may be determined based on an effective dose of the pharmaceutical composition, but in the case of long term administration for the purpose of health or hygiene or to control health, the effective dose may be within this range of less. Also, the effective component has no problem in terms of safety, and thus it is certain that the effective dose may be used within this range or more.

A type of the health food is not particularly limited, and examples thereof may include meats, sausages, breads, chocolates, candies, snacks, cookies, pizza, Ramen or other noodles, gums, dairy products including ice cream, soups, beverages, teas, drinks, alcohol drinks, and vitamin complexes.

### MODE OF THE INVENTIVE CONCEPT

Hereinafter, the present invention will be described in detail with reference to the following examples. However, the examples are only for the purpose of illustrations only, not intended to limit the scope of the invention. It should be understood that the examples are provided for a more definite explanation to an ordinary person skilled in the art.

The following Reference Example is described to provide a reference example that commonly applies to each of embodiments according to the present invention.

### Reference Example

### 1. Experiment Animal and Reagent

A methionine-choline deficient (MCD) feed to induce steatohepatitis was purchased from Dyets (Bethlehem, Pa., USA). Alkoxy glycerol (batyl alcohol), as a plasmalogen precursor, was purchased from Sigma (St Louis, Mo., USA).

2% BSA was prepared in a DMEM:F12 medium, and plasmalogen precursor alkoxy glycerol dissolved in ethanol was added thereto After incubation at 70° C. for 20 minutes, the mixture was inoculated in a manner of observing crystal disappearance. 7-week-old C57/BL6 male mice were brought in to an experimental animal room, stabilized for 7 days, and then separated into a general feed group, as a control group, an MCD diet group (an MCDD group), and a group taken a mixture of MCD diet and 100 mg/kg/day of plasmalogen precursor alkoxy glycerol (an AG group), where each of the groups had six mice. The test feeds were taken for the total of 6 weeks, and the mice were allowed to only take water for the following 12 hours. Then, blood samples were obtained from the mice, and liver tissues were harvested from each of the mice. The harvested liver tissues were instantly rapid-frozen in dry ice and stored with plasma in a freezer at −70° C.

### Example 1

### Blood Sample Measurement

Blood samples were obtained from venae cavae of the experimental animals, the plasma was separated from the blood samples by using a centrifuge (at 300 rpm for 15 minutes), and alanine aminotransferase (ALT) of the plasma was measured by using a kit of Labs Biotechnology (London, Canada). The ALT is a marker that sensitively reflects degrees of inflammation and damage on liver cells, which is widely used to figure out a status of various liver diseases such as hepatitis or liver cirrhosis, as well as hepatic steatosis. In regard of the change in plasma ALT per experimental group, the AG group, which was administered with MCD diet together with 100 mg/kg/day of plasmalogen precursor alkoxy glycerol, showed significantly improvement compared to that of the MCDD group as shown in FIG. 1.

### Example 2

### Neutral Fats in Liver Tissue Measurement

Neutral fats in the liver tissue was measured by using a triglyceride (GPO-Trinder) kit purchased from Sigma. The titrated liver tissue and plasma were allowed to react with an extraction buffer at room temperature for 4 hours, 1N H₂SO₄ was added thereto, centrifuged at 100 rpm for 10 minutes, and 100 mg of Na₂S₂O₂ was added to a remaining solution after removing a supernatant the centrifuged resultant, and the resultant was centrifuged at 1000 rpm for 5 minutes. Then, a supernatant was obtained therefrom, 0.5 g of silicic acid was added thereto, allowed to react at room temperature for 5 minutes, centrifuged at 1000 rpm for 10 minutes, the supernatant was evaporated with N₂ gas to obtain a sample, and the sample was dissolved with isopropanol. Then, a triglyceride reagent A was added thereto, allowed to react at room temperature for 5 minutes, and the measurement was performed by using an Emax precision micro plate reader (Molecular Devices, Sunnyvale, Calif.) at 540 nm. A change in an amount of neutral fats of liver tissues per experimental group is the same as shown in FIG. 2, as resulted in regard of the plasma ALT, the AG group, which was administered with MCD diet together with 100 mg/kg/day of plasmalogen precursor alkoxy glycerol, showed significantly improvement results.

### Example 3

### Inflammation Change of Liver Tissue

In order to confirm change in an inflammation degree of liver tissues, gene expression of a tumor necrosis factor (TNF-α) and monocyte chemotactic protein-1 (MCP-1), which are typical inflammatory cytokines, were confirmed by real-time PCR. As a result, referring to FIG. 3, as resulted in regard of the plasma ALT and the amounts of neutral fats in the liver, it was confirmed that administration of 100 mg/kg/day of plasmalogen precursor alkoxy glycerol suppressed expression of inflammatory cytokine genes that was increased by MCD diet.

### Example 4

### Histological Observation

A part of the harvested liver tissues were fixed with 10% neutral formalin solution, and embedded in paraffin, and formaldehyde was washed in running water for 12 hours or more. Then, the resultant was step-wisely washed with 60% ethanol for 1 hour, 70% ethanol for 1 hour, 80% ethanol for 1 hour, 90% ethanol for 1 hour, 5% ethanol for 1 hour, and 100% ethanol for 1 hour. Thereafter, the total of three transparent processes and two penetration processes were each performed for 1 hour in xylene, and the resultant underwent an embedding process to prepare a segment having a thickness of about 4 μm.

The segment was died with hematoxylin-eosin (H&E) and trichrome C, and examined and analyzed by using an optical microscope.

Representative H&E and trichrome C dye images of the control group, MCDD group, and AG group are as shown in FIG. 4, and it may be observed that steatohepatitis was relieved to the similar level with that of the control group by the administration of 100 mg/kg/day of plasmalogen precursor alkoxy glycerol.

### Example 5

### Fatty Acid Oxidation Measurement

In order to measure fatty acid oxidation in the liver tissue, ¹⁴CO₂ production amounts from ¹⁴C-palmitate (NEN Life Sciences, Boston, Mass., USA) was measured and analyzed.

In this regard, a reaction buffer including 0.2 mM of palmitate (1-¹⁴C-palmitate at 0.5 μCi/ml) was added to 50 μl of a liver titrate, and this was allowed to react at 30° C. for 2 hours.

Then, 50 μl of 4 N sulfuric acid, as a stopping buffer, was added thereto, and the mixture was allowed to react for 2 hours, and then a ¹⁴CO₂ production amount was measured. In order to examine fatty acid oxidation of peroxisome, which is in charge of fatty acid beta oxidation, other than mitochondria, 12.5 μM of rotenone, as a mitochondria suppressor, and 100 μM of antimycin were added to a reaction buffer together with 0.2 mM of palmitate (1-¹⁴C palmitate at 0.5 μCi/ml), and the reaction mixture was allowed to react at 30° C. for 2 hours. Then, in the same manner, 50 μl of 4 N sulfuric acid, as a stopping buffer, was added thereto, allowed to react for 2 hours, and then a ¹⁴CO₂ production amount was measured.

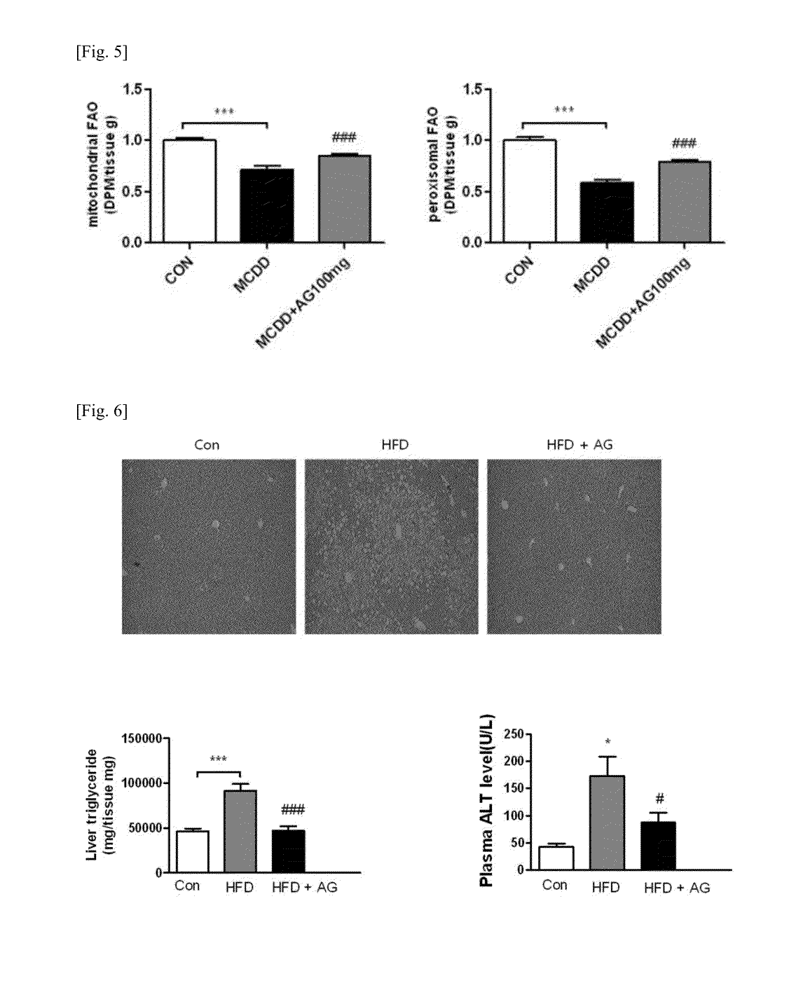

As the result, fatty acid oxidation of peroxisome in the liver tissues per experimental group was confirmed as shown in FIG. 5.

### Example 6

### Confirmation of Plasmalogen Precursor Effect in High Fat Diet Hepatic Steatosis Animal Model

Although a MCDD animal model is a model that represents nonalcoholic steatohepatitis occurrence, the MCDD animal model cannot represent a risk factor that accompanies metabolic syndrome. In order to overcome such limitation, liver tissues of mice that were allowed to take high fat diet (HFD) as a general animal model with respect to hepatic steatosis were used for the comparison.

The animal model only had simple hepatic steatosis without inflammation unlike the MCDD group.

Alkoxy glycerol, as a plasmalogen precursor, was added to the HFD feed that induces obesity and non-alcoholic hepatic steatosis, and the feed was administrated to the animal model, and the change was confirmed.

First, the control group (CON) was prepared by allowing 7-week-old C57/BL6 male mice to take a general feed including 7% of fat, 18% of protein, and 75% of carbohydrate, and the HFD group was prepared by allowing 7-week-old C57/BL6 male mice to freely take a HFD feed including 60% of fat, 18% of protein, and 22% of carbohydrate.

Also, the AG group (AG-200) was allowed to take the HFD mixed with 200 mg/kg/day of alkoxy glycerol. The experimental groups were allowed to take the corresponding feeds for the total of 12 weeks, and liver histological changes in the livers were confirmed in the same manner in Example 4, and the plasma ALT levels were confirmed in the same manner as in Example 1.

As the result, as shown in FIG. 6, it was confirmed that the AG group (AG-200) administered with the HFD together with alkoxy glycerol showed significant improvement in terms of hepatic steatosis occurrence and lipid content in the liver tissues and remarkable reduction in the plasma ALT level, which is a liver damage marker.

Hereinafter, formulation examples of the pharmaceutical composition of the present invention will be described, but the examples are for the purpose of illustrations only, not intended to limit the scope of the invention.

### Formulation Example 1

### Preparation of Injection

10 mg of plasmalogen, 3.0 mg of sodium metabisulfite, 0.8 mg of methylparaben, 0.1 mg of propylparaben, and an appropriate amount of sterilized distilled water for injection were prepared by using a general method to have the total volume of 2 ml, and the resultant was filled in an 2 ml ample and sterilized to prepare an injection.

### Formulation Example 2

### Preparation of Tablet

10 mg of batyl alcohol, 100 mg of lactose, 100 mg of starch, and an appropriate amount of magnesium stearic acid were mixed, and molded into a table by using a common tablet preparation method.

### Formulation Example 3

### Preparation of Capsule

10 mg of batyl alcohol, 50 mg of lactose, 50 mg of starch, 2 mg of talc, and an appropriate amount of magnesium stearic acid were mixed, and filled in a gelatin capsule by using a common capsule preparation method to prepare a capsule.

Hereinafter, preparation examples of health food by using the plasmalogen or an analog thereof according to the present invention will be described, but the examples are for the purpose of illustrations only, not intended to limit the scope of the invention.

### Preparation Example 1

### Preparation of Health Food

10 mg of batyl alcohol, an appropriate amount of vitamin mixture (70 μg of vitamin A acetate, 1.0 mg of vitamin E, 0.13 mg of vitamin B1, 0.15 mg of vitamin B2, 0.5 mg of vitamin B6, 0.2 μg of vitamin B12, 10 mg of vitamin C, 10 μg of biotin, 1.7 mg of nicotinic acid amide, 50 μg of folic acid, and 0.5 mg of calcium pantothenate), and an appropriate amount of inorganic mixture (1.75 mg of iron (II) sulfate, 0.82 mg of zinc oxide, 25.3 mg of magnesium carbonate, 15 mg of potassium phosphate, 55 mg of calcium phosphate, 90 mg of potassium citric acid, 100 mg of calcium carbonate, and 24.8 mg of magnesium chloride) were mixed, granules were prepared, and health food was prepared by using a common method.

### Preparation Example 2

### Preparation of Health Drink

10 mg of shark liver oil, 1000 mg of citric acid, 100 g of oligoscaccharide, 2 g of green plum extract, 1 g of taurine, and purified water at an amount that accounts for making the total volume of 900 ml, and the ingredients were mixed according to a common health drink preparation method, stirred and heated for about 1 hour at 85° C., and the resulting solution was filtered. The filtered solution was contained in a 2 L-container sealed and sterilized, and stored in a refrigerator.

1. A method of preventing or treating non-alcoholic fatty liver diseases or hepatic steatosis in a subject in need thereof, comprising:
providing a pharmaceutical composition comprising a therapeutically effective amount of a plasmalogen precursor alkoxy glycerol or shark liver oil containing an alkoxy glycerol as an effective ingredient and
administering the pharmaceutical composition to the subject, wherein the non-alcoholic fatty liver diseases or hepatic steatosis is prevented or treated.

2. The method of claim 1, wherein the plasmalogen precursor alkoxy glycerol comprises the alkoxy glycerol having a C16-C18 fatty alcohol linked to a glycerol backbone via an ether bond.

3. The method of claim 2, wherein the plasmalogen precursor alkoxy glycerol further comprises is selected from the group consisting of a batyl alcohol.

4. (canceled)

5. The method of claim 1, wherein the pharmaceutical composition decreases accumulation of neutral fats, suppresses expression of inflammatory cytokine genes, and increases fatty acid oxidation of peroxisome.

6. The method of claim 1, wherein the plasmalogen precursor alkoxy glycerol is contained at an amount in a range of about 0.01 part to about 90 parts by weight based on 100 parts by weight of the total weight of the pharmaceutical composition.

7. (canceled)

8. A method of relieving or preventing non-alcoholic fatty liver diseases or hepatic steatosis, comprising:
administering a subject a health food composition comprising a plasmalogen precursor alkoxy glycerol or shark liver oil containing an alkoxy glycerol as an effective component.
